(19) United States
(12) Patent Application Publication
(10) Pub. No.: US 2020/0363372 A1
(43) Pub. Date: Nov. 19, 2020

(54) ANALYSIS SYSTEM, FECES ODOR GAS ANALYSIS SYSTEM, AND EXHALED GAS ANALYSIS SYSTEM

(71) Applicants: SHINICHI KUBOTA, OSAKA (JP); KATSUYA UJIMOTO, HYOGO (JP); KUNIHIRO TAN, HYOGO (JP)

(72) Inventors: SHINICHI KUBOTA, OSAKA (JP); KATSUYA UJIMOTO, HYOGO (JP); KUNIHIRO TAN, HYOGO (JP)

(73) Assignee: Ricoh Company, Ltd., Tokyo (JP)

(21) Appl. No.: 16/856,243

(22) Filed: Apr. 23, 2020

(30) Foreign Application Priority Data
May 16, 2019 (JP) 2019-093221

(51) Int. Cl. G01N 27/62; G01N 27/70; G01N 33/497

(52) U.S. Cl. CPC G01N 27/622; G01N 33/497; G01N 27/70

(57) ABSTRACT

An analysis system includes: an ionizer to ionize sample gas so as to have a first polarity; a detection circuit to detect at least a part of ions of the sample gas; a housing accommodating the ionizer and the detection circuit; and a voltage generation circuit to apply a voltage having the first polarity to the housing to generate an electric field inside the housing between the ionizer and the detection circuit, the electric field having a first component in a first direction perpendicular to a second direction parallel to a flow direction of the sample gas and a second component in a third direction perpendicular to the first direction and the second direction.

### CROSS-REFERENCE TO RELATED APPLICATIONS

This patent application is based on and claims priority pursuant to 35 U.S.C. § 119(a) to Japanese Patent Application No. 2019-093221, filed on May 16, 2019 in the Japan Patent Office, the entire disclosure of which is hereby incorporated by reference herein.

### BACKGROUND

### Technical Field

The present invention relates to an analysis system, a feces odor gas analysis system, and an exhaled gas analysis system.

### Description of the Related Art

Various studies have been conducted on the detection and analysis of molecules using the Field Asymmetric Ion Mobility Spectrometry (FAIMS) system. The FAIMS system includes an ion filter including a pair of electrodes to which an asymmetric alternating current (AC) signal is applied. When ionized gas molecules flow through the ion filter, the ions are sorted depending on the difference in mobility. The ion having passed through the ion filter is caused to collide with an ion detection electrode, and the current generated by the ion detection electrode is detected so that the gas component may be identified.

According to a background art, there is the FAIMS system that uses a pair of confinement electrodes to guide an ionized sample to an ion filter.

However, it is difficult to obtain the sufficient detection sensitivity with the FAIMS system according to the background art.

### SUMMARY

Example embodiments include an analysis system including: an ionizer to ionize sample gas so as to have a first polarity; a detection circuit to detect at least a part of ions of the sample gas; a housing accommodating the ionizer and the detection circuit; and a voltage generation circuit to apply a voltage having the first polarity to the housing to generate an electric field inside the housing between the ionizer and the detection circuit, the electric field having a first component in a first direction perpendicular to a second direction parallel to a flow direction of the sample gas and a second component in a third direction perpendicular to the first direction and the second direction.

### BRIEF DESCRIPTION OF THE SEVERAL VIEWS OF THE DRAWINGS

A more complete appreciation of the disclosure and many of the attendant advantages and features thereof can be readily obtained and understood from the following detailed description with reference to the accompanying drawings, wherein:

FIG. 1 is a diagram illustrating the trajectory of movement of ions in an example of an ion detection device according to an embodiment of the present invention;

FIG. 2 is a graph illustrating an electric field intensity dependence of mobility of the ion according to an embodiment of the present invention;

FIG. 3 is a graph illustrating an example of an electric field waveform generated by an ion filter according to an embodiment of the present invention;

FIG. 4 is a schematic view illustrating an example of the configuration of an analysis system according to a first embodiment of the present invention;

FIG. 5 is a cross-sectional view illustrating an example of the configuration of the analysis system according to the first embodiment;

FIG. 6 is a graph illustrating an example of an offset voltage and a compensation voltage when sample gas is positively ionized according to an embodiment of the present invention;

FIG. 7 is a graph illustrating an example of an offset voltage and a compensation voltage when sample gas is negatively ionized according to an embodiment of the present invention;

FIG. 8 is a graph illustrating an example of an asymmetric voltage waveform for producing the example of the electric field waveform illustrated in FIG. 3 according to an embodiment of the present invention;

FIG. 9 is a graph illustrating another example of the asymmetric voltage waveform for producing the example of the electric field waveform illustrated in FIG. 3 according to an embodiment of the present invention;

FIG. 10 is a schematic view illustrating an example of the configuration of an analysis system according to a second embodiment of the present invention;

FIG. 11 is a cross-sectional view illustrating an example of the configuration of the analysis system according to the second embodiment;

FIG. 12 is a schematic view illustrating an example of the configuration of an analysis system according to a third embodiment of the present invention;

FIG. 13 is a schematic view illustrating a device used in an experiment according to an embodiment of the present invention;

FIG. 14 is a graph illustrating results of the experiment according to an embodiment of the present invention; and

FIG. 15 is a schematic view illustrating another example of the ion filter according to an embodiment of the present invention.

The accompanying drawings are intended to depict embodiments of the present invention and should not be interpreted to limit the scope thereof. The accompanying drawings are not to be considered as drawn to scale unless explicitly noted.

### DETAILED DESCRIPTION

The terminology used herein is for the purpose of describing particular embodiments only and is not intended to be limiting of the present invention. As used herein, the singular forms “a”, “an” and “the” are intended to include the plural forms as well, unless the context clearly indicates otherwise.

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring to the accompanying drawings, an embodiment of the present disclosure is described below. In the description and the drawings, the components having substantially the same function and configuration are denoted by the same reference numeral, and duplicated descriptions may be omitted.

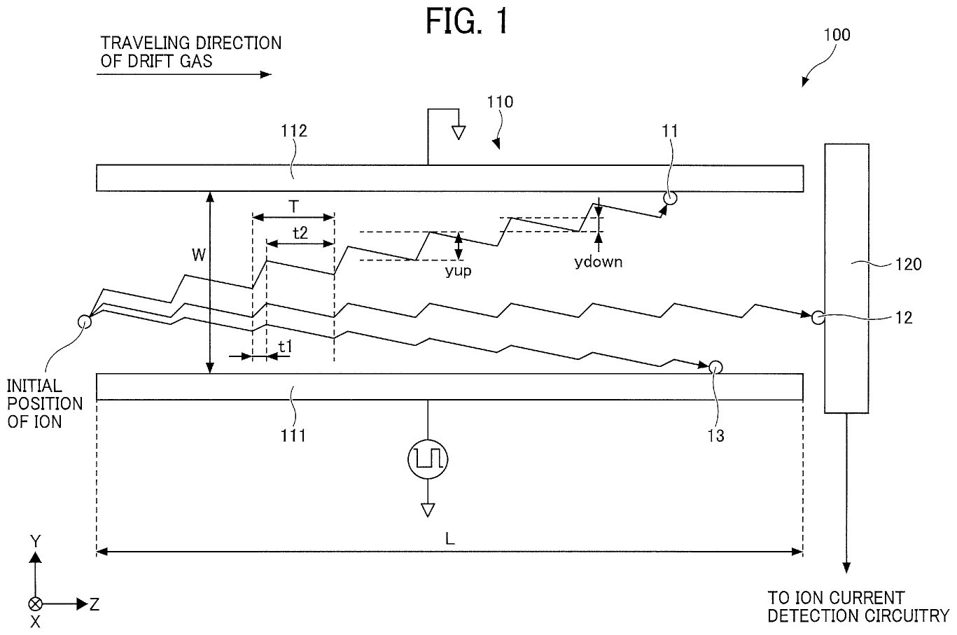

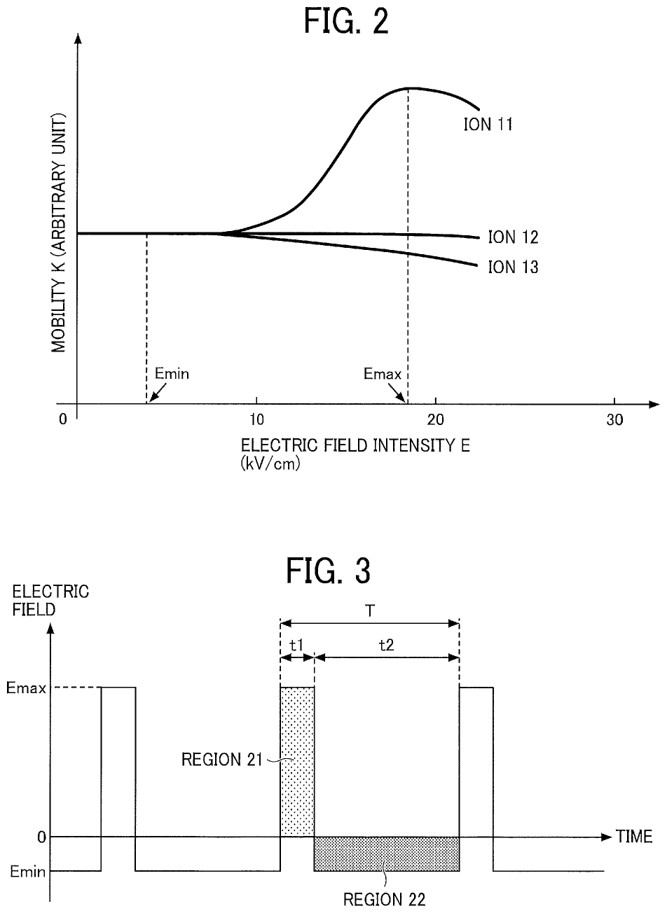

Ion Detection Device First, the configuration and the basic principle of an ion detection device 100 used in the FAIMS system is described. FIG. 1 is a diagram illustrating the trajectory of movement of ions in an example of the ion detection device 100. FIG. 2 is a graph illustrating the electric field intensity dependence of mobility of the ion. FIG. 3 is a graph illustrating an example of the electric field waveform generated by an ion filter.

As illustrated in FIG. 1, the ion detection device 100 includes an ion filter 110 including a first electrode 111 and a second electrode 112 that are opposed to each other, and an ion detection electrode 120 with which passing ions, which have passed through the ion filter 110, collide.

An ion current detection circuit is coupled to the ion detection electrode 120 while the ion detection device 100 is used. The electric current corresponding to the number of ions that have collided with the ion detection electrode 120 are generated, and the electric current is detected by the ion current detection circuit. Here, the XYZ three-dimensional orthogonal coordinate system is used; the traveling direction of the molecule to be analyzed is in the +Z direction, the direction viewed from the first electrode 111 toward the second electrode 112 is in the +Y direction, and the direction perpendicular to the +Y direction and the +Z direction is in the +X direction.

In the environment of an electric field E, an ion moves at a moving velocity V represented by the following Equation (1). Here, K is the mobility of the ion.

V=K×E (1)

The mobility of an ion has the electric field intensity dependence. The electric field intensity dependence is different depending on the type of ion. FIG. 2 illustrates, for example, the electric field intensity dependence of the mobility with regard to three different types of ions (an ion 11, an ion 12, and an ion 13). In FIG. 2, for simplicity, the mobility of each ion is normalized so as to be equal at an electric field intensity of zero.

The mobilities of the three ions (the ion 11, the ion 12, and the ion 13) have almost no change at a low electric field intensity of 9 kV/cm or less. With an electric field intensity of approximately 10 kV/cm or more, the inherent property of the type of ion appears in the mobility. The mobility of the ion 11 largely increases in accordance with an increase in the electric field intensity and reaches a maximum at a positive high electric field (Emax). The mobility of the ion 12 hardly changes regardless of the electric field intensity. The mobility of the ion 13 gradually decreases. Thus, each of the three exhibits a different property. The ion filter 110 uses the difference in the mobility between a low electric field intensity and a high electric field intensity to sort ions.

FIG. 1 illustrates the trajectories of movement of the three ions (the ion 11, the ion 12, and the ion 13) between the first electrode 111 and the second electrode 112 of the ion filter 110. For the sake of simplicity and convenience, the first electrode 111 and the second electrode 112 are parallel flat plates including a conductor.

As the waveform of the electric field generated between the first electrode 111 and the second electrode 112 is an asymmetric electric field waveform, an arbitrary ion (the ion 12 in FIG. 1) may reach the ion detection electrode 120.

FIG. 3 illustrates an example of the electric field waveform generated between the first electrode 111 and the second electrode 112. The electric field waveform has a positive high electric field (Emax) and a negative low electric field (Emin) that are alternately repeated. The period (t1) of the high electric field is shorter than the period (t2) of the low electric field, and the ratio between t1 and t2 is 1:3 to 1:5. Thus, the electric field waveform is asymmetric with respect to a vertical direction. The asymmetric electric field waveform is set such that the time-averaged electric field is zero and the following Equation (2) is satisfied.

|Emax|×t1=|Emin|×t2 (2)

That is, the asymmetric electric field waveform is set such that the size of a region 21 and the size of a region 22 in FIG. 3 are identical.

As represented by the following Equation (3), the value of |Emax|×t1 and the value of |Emin|×t2 are β below.

|Emax|×t1=|Emin|×t2=β (3)

The velocity (Vup) at which an ion moves in the Y-axis direction during the period (t1) of the high electric field is represented by the following Equation (4). Here, K(Emax) is the mobility of the ion in the high electric field (Emax).

Vup=K(Emax)×|Emax| (4)

For example, when |Emax| is approximately 10 kV/cm or more, the mobility of each of the three ions (the ion 11, the ion 12, and the ion 13) is different, and therefore the moving velocity (Vup) of each of the three ions is different. That is, as illustrated in FIG. 1, during the period (t1) of the high electric field, the inclinations of the movement trajectories of the three ions are different from each other.

The displacement (yup), which is the distance by which the ion has moved in the Y-axis direction during the period (t1) of the high electric field, is represented by the following Equation (5).

yup=Vup×t1 (5)

The velocity (Vdown) at which the ion moves in the Y-axis direction during the period (t2) of the low electric field is represented by the following Equation (6). Here, K(Emin) is the mobility of the ion in the low electric field (Emin).

Vdown=K(Emin)×|Emin| (6)

For example, when |Emin| is approximately 5 kV/cm or less, the mobilities of the three ions (the ion 11, the ion 12, and the ion 13) are almost the same, and therefore the moving velocities (Vdown) of the three ions are almost the same. That is, as illustrated in FIG. 1, during the period (t2) of the low electric field, the inclinations of the movement trajectories of the three ions are almost the same.

The displacement (ydown), which is the distance by which the ion has moved in the Y-axis direction during the period (t2) of the low electric field, is represented by the following Equation (7).

ydown=Vdown×t2 (7)

In one cycle (T) of the asymmetric electric field waveform, while the ion moves in the +Z direction, the ion moves in the +Y direction during the period (t1) and moves in the −Y direction during the period (t2).

As illustrated in FIG. 1, the ions are classified into: an ion (the ion 11) that repeats a zigzag movement to move toward the first electrode 111; an ion (the ion 13) that repeats a zigzag movement to move toward the second electrode 112; and an ion (the ion 12) that moves toward the ion detection electrode 120 due to the balance between the displacement in +Y direction and the displacement in the −Y direction.

The average displacement (ΔyRF) of the ion in the Y-axis direction during one cycle (T) of the asymmetric electric field waveform is represented by the following Equation (8).

$$Δ yRF = yup + ydown = K ( E max ) \times \vert Emax \vert \times t 1 - K ( E min ) \times \vert Emin \vert \times t 2 \qquad ( 8 )$$

The above-described Equation (8) may be represented as the following Equation (9) using the above Equation (3).

ΔyRF=β{(K(Emax)−K(min)} (9)

Here, when K(Emax)−K(min) is replaced with ΔK, the above Equation (9) is represented as the following Equation (10).

ΔyRF=βΔK (10)

Here, β is a constant determined due to the asymmetric electric field applied between the first electrode 111 and the second electrode 112. Therefore, the displacement of the ion in the Y-axis direction per one cycle (T) of the asymmetric electric field waveform depends on ΔK that is the difference between the mobility in the low electric field (Emin) and the mobility in the high electric field (Emax).

Assuming that the carrier gas moves an ion in the Z-axis direction, the displacement (Y) of the ion in the Y-axis direction when the ion stays between the first electrode 111 and the second electrode 112 is represented by the following Equation (11). Here, tres is the average time (the average ion stay time) during which the ion stays between the first electrode 111 and the second electrode 112.

$$\begin{matrix}[ Equation ( 1 ) ] & \\ Y = \frac{Δ yRF}{( t 1 + t 2 )} \times tres = \frac{β Δ K}{T} \times tres & ( 11 )\end{matrix}$$

The average ion stay time tres is represented by the following Equation (12). Here, A is the cross-sectional area of the ion path in the ion filter 110, L is the electrode length (electrode depth) in the Z-axis direction, and Q is the volume flow rate of the carrier gas. V is the volume (=A×L) of the ion filter 110. The cross-sectional area A of the ion path is a product of the width W (FIG. 1) between the first electrode 111 and the second electrode 112, and a X-direction length of the first electrode 111 and the second electrode 112.

$$\begin{matrix}[ Equation ( 2 ) ] & \\ tres = \frac{AL}{Q} = \frac{V}{Q} & ( 12 )\end{matrix}$$

The above-described Equation (11) may be represented as the following Equation (13) using Equation (12) and Equation (3) described above. Here, D is the duty of the asymmetric electric field waveform, and D=t1/T.

$$\begin{matrix}[ Equation ( 3 ) ] & \\ Y = \frac{Δ K \times E max \times V \times D}{Q} & ( 13 )\end{matrix}$$

When the same value is used for all types of ions with regard to the high electric field (Emax) in the asymmetric electric field waveform, the volume (V) of the ion path in the ion filter 110, the duty (D) of the asymmetric electric field waveform, and the volume flow rate (Q) of the carrier gas, it is understood, from the above-described Equation (13), that the displacement (Y) is proportional to the difference ΔK between the mobility in the low electric field (Emin) and the mobility in the high electric field (Emax) that are unique to each type of ion.

In FIG. 1, the displacement (Y) of the ion 12 is the smallest, and the ion 12 reaches the ion detection electrode 120. By changing the duty (D), the ion having ΔK different from that of the ion 12 may reach the ion detection electrode 120. Furthermore, by changing the duty (D) little by little, it is possible to detect the presence or absence or the number of various ions having different ΔK.

Changing the dispersion voltage (VDF), which is the difference between the high electric field (Emax) and the low electric field (Emin), while keeping the constant duty (D) makes it possible to detect the presence or absence or the number of various ions having different ΔK.

One of the methods for detecting various types of ions having different ΔK with the ion detection device 100 is the method for superimposing a low-intensity direct-current (DC) electric field on an asymmetric electric field waveform. This method may change the degree of displacement in the Y-axis direction during the period (t1) and the period (t2). Thus, it is possible to continuously change the type of ion that may reach the ion detection electrode 120 without being in contact with at least one of the first electrode 111 and the second electrode 112. The DC electric field superimposed on the asymmetric electric field waveform is called a compensation voltage (CV). In this method, sweeping the compensation voltage enables the detection of the presence or absence or the number of various types of ions having different ΔK.

The data on the number of detected ions is acquired under the conditions in which various values of the dispersion voltage and the compensation voltage described above are combined, whereby it is possible to analyze the presence or absence of various types of ions more accurately.

If an ion comes into contact with at least one of the first electrode 111 and the second electrode 112 before reaching the ion detection electrode 120, the ion is neutralized so as to be no longer an ion and be undetectable.

Thus, the ion detection device 100 may selectively detect ions.

When the ionized molecules are supplied to the ion filter 110 by an ionizer, or the like, the ionized molecules are sometimes absorbed by a housing, or the like, which accommodates the ion filter 110, before reaching the ion filter 110. Due to this absorption, the number of ions reaching the ion detection electrode 120 decreases, which may result in insufficient detection sensitivity.

### First Embodiment

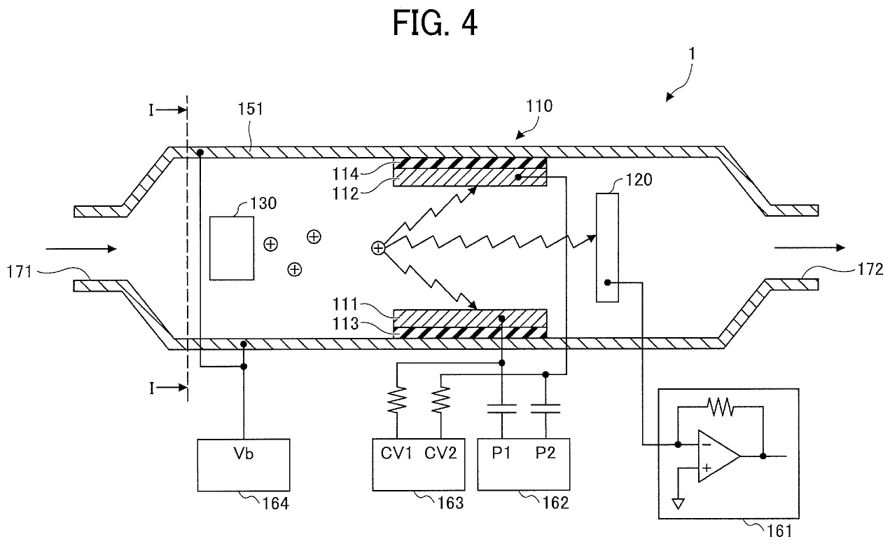

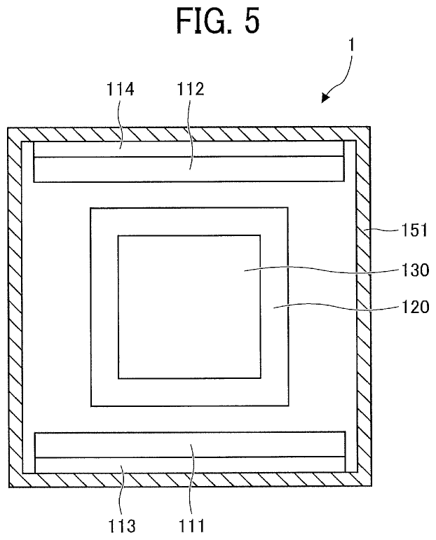

Next, a first embodiment of the present invention is described. The first embodiment relates to an analysis system 1 to which the FAIMS system is applied. FIG. 4 is a schematic view illustrating an example of the configuration of the analysis system 1 according to the first embodiment. FIG. 5 is a cross-sectional view illustrating an example of the configuration of the analysis system 1 according to the first embodiment. FIG. 5 corresponds to the cross-sectional view taken through the line I-I in FIG. 4.

The analysis system 1 according to the first embodiment includes an ionizer 130, the ion filter 110, and the ion detection electrode 120. The ionizer 130, the ion filter 110, and the ion detection electrode 120 are accommodated in a housing 151. The ionizer 130, the ion filter 110, and the ion detection electrode 120 are sequentially arranged in this order from an inlet 171 toward an outlet 172 of the housing 151. The ion filter 110 includes the first electrode 111 and the second electrode 112. The housing 151 is conductive. The housing 151 includes, for example, metal. An insulator 113 is provided between the housing 151 and the first electrode 111 to electrically isolate the housing 151 and the first electrode 111 from each other. An insulator 114 is provided between the housing 151 and the second electrode 112 to electrically isolate the housing 151 and the second electrode 112 from each other. The analysis system 1 further includes an ion current detection circuit 161, an asymmetric waveform signal generation circuitry 162, a compensation voltage generation circuitry 163, and a bias voltage generation circuitry 164. The direction viewed from the first electrode 111 to the second electrode 112 is an example of a first direction. The direction from the inlet 171 to the outlet 172 is an example of a second direction. The direction perpendicular to the first direction and the second direction is an example of a third direction.

The ionizer 130 ionizes sample gas by using, for example, actinogen, corona discharge, ultraviolet rays, or catalyst. The ionizer 130 is an example of an ionizing means. The ion filter 110 selectively allows the passage of part of the ionized sample gas. The current corresponding to the number of ions having collided with the ion detection electrode 120 is generated, and the current is detected by the ion current detection circuit 161.

The asymmetric waveform signal generation circuitry 162 feeds the signal having an asymmetric voltage waveform for producing the example of the asymmetric electric field waveform illustrated in FIG. 3 to the first electrode 111 and the second electrode 112. The asymmetric waveform signal generation circuitry 162 feeds a signal P1 to the first electrode 111. The asymmetric waveform signal generation circuitry 162 feeds a signal P2 to the second electrode 112. The compensation voltage generation circuitry 163 feeds the offset voltage and the compensation voltage to the first electrode 111 and the second electrode 112. The compensation voltage generation circuitry 163 feeds a signal CV1 to the first electrode 111. The compensation voltage generation circuitry 163 feeds a signal CV2 to the second electrode 112.

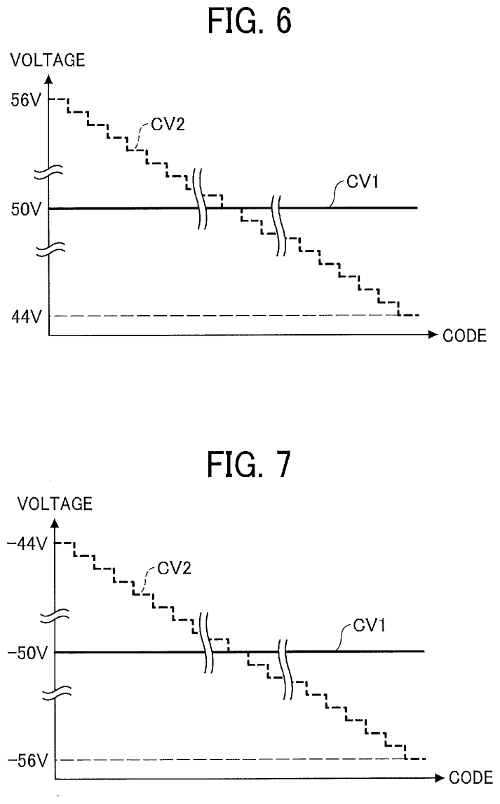

Here, the offset voltage and the compensation voltage are described. FIG. 6 is a graph illustrating an example of the offset voltage and the compensation voltage when the sample gas is positively ionized. FIG. 7 is a graph illustrating an example of the offset voltage and the compensation voltage when the sample gas is negatively ionized.

When the sample gas is positively ionized, for example, the voltage of the signal CV1 is the constant (here, 50 V) offset voltage. When the sample gas is negatively ionized, for example, the voltage of the signal CV1 is the constant (here, −50 V) offset voltage. For example, the voltage of the signal CV2 is the voltage obtained by superimposing the compensation voltage of ±6 V at maximum, which is changed together with the code, on the same offset voltage as that of the signal CV1.

The range of the compensation voltage is not limited to ±6 V but may be any range such as ±10 V or ±2 V depending on the purpose of analysis. Furthermore, the step voltage to be changed may be set to any value in consideration of the desired resolution.

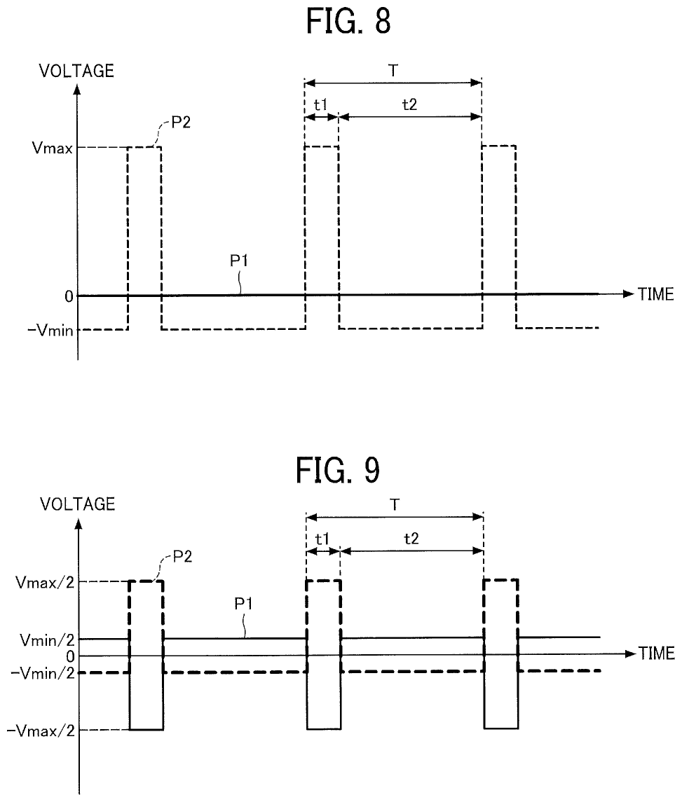

Next, the signal having an asymmetric voltage waveform is described. FIG. 8 is a graph illustrating an example of the asymmetric voltage waveform for producing the example of the electric field waveform illustrated in FIG. 3. In the example illustrated in FIG. 8, the signal P1 having the constant voltage (here, 0 V) is fed to the first electrode 111, and the high-frequency waveform signal P2 having the cycle T, the pulse width (period) t1, and the pulse width (period) t2, which are identical to those of the asymmetric waveform electric field illustrated in FIG. 3, is fed to the second electrode 112. The amplitude of the high-frequency waveform signal P2 is Vmax to −Vmin that correspond to Emax to Emin.

As illustrated in FIG. 4, the signal P1 and the signal P2 are fed to the first electrode 111 and the second electrode 112, respectively, via capacitors. Therefore, the AC component of an asymmetric voltage waveform is transmitted to the first electrode 111 and the second electrode 112. The signal CV1 and the signal CV2 are fed to the first electrode 111 and the second electrode 112, respectively, via resistors. Therefore, the average voltage of the signal applied to the first electrode 111 is the voltage of the signal CV1, and the average voltage of the signal applied to the second electrode 112 is the voltage of the signal CV2.

The bias voltage generation circuitry 164 applies a bias voltage Vb to the housing 151. The bias voltage generation circuitry 164 applies the positive bias voltage Vb, for example, +50 V, to the housing 151 when the sample gas is positively ionized. The bias voltage generation circuitry 164 applies the negative bias voltage Vb, for example, −50 V, to the housing 151 when the sample gas is negatively ionized. The bias voltage generation circuitry 164 is an example of a voltage applying means.

In the analysis system 1 according to the first embodiment, the sample gas is introduced into the housing 151 through the inlet 171. The ionizer 130 ionizes sample gas, which has been introduced into the housing 151. Due to the effect of the ion filter 110, at least part of the ionized sample gas reaches the ion detection electrode 120, the current corresponding to the number of ions having collided with the ion detection electrode 120 is generated, and the current is detected by the ion current detection circuit 161. The sample gas is discharged to the outside through the outlet 172.

When the ionizer 130 positively ionizes the sample gas, the bias voltage generation circuit 164 applies the positive bias voltage Vb to the housing 151. When the ionizer 130 negatively ionizes the sample gas, the bias voltage generation circuit 164 applies the negative bias voltage Vb to the housing 151. As illustrated in FIG. 5, when viewed along the flow path of the sample gas, the housing 151 exists on both sides in the first direction and both sides in the third direction. The electric field generated in the neighborhood of the housing 151 has both a first component in the first direction and a second component in the third direction. As the polarity of the ion of the sample gas is the same as the polarity of the bias voltage Vb, the ion of the sample gas receives an electric repulsive force from the housing 151, is likely to flow through the center of the space inside the housing 151, and is unlikely to come into contact with the housing 151. For this reason, according to the first embodiment, the sufficient number of ions may reach the ion detection electrode 120, which may result in an improvement in the detection sensitivity.

In a case where the ionizer 130 ionizes the sample gas due to the corona discharge, as the bias voltage Vb is closer to the voltage (e.g., +5 kV or −5 kV) of a corona discharge electrode, the contact with the housing 151 is easily suppressed. Conversely, if the discharge occurs due to the too high bias voltage Vb, the difficulty easily arises in the accurate analysis. Typically, in the case of approximately several tens of V to 100 V, no discharge occurs, and the bias voltage generation circuit 164 is easily implemented. Therefore, the bias voltage Vb may be approximately several tens of V to ±100 V.

According to the experiment performed by the inventors of the present application, when the following relationship is established among the voltage of the discharge electrode of the ionizer 130, the bias voltage Vb, the offset voltage of the ion filter 110, and the potential of the ion detection electrode 120, a particularly desirable sensitivity may be obtained.

|The voltage of the discharge electrode of the ionizer 130|>|the bias voltage Vb|≥|the offset voltage of the ion filter 110|>|the potential of the ion detection electrode 120|

The cross-sectional shape of the flow path formed by the inner wall surfaces of the housing 151 for the sample gas is not limited to a square (referring to FIG. 5) and may be another shape such as a circle. The housing 151 does not need to be integrally formed, and a plurality of parts may be combined in the circumferential direction of the housing 151, with a screw, an adhesive, etc. The housing 151 may be formed in a annular shape continuous in the circumferential direction of the housing 151. That is, the housing 151 may entirely surround the flow path of the sample gas in four directions.

The signal having an asymmetric voltage waveform is not limited to the example illustrated in FIG. 8. FIG. 9 is a graph illustrating another example of the asymmetric voltage waveform for producing the example of the electric field waveform illustrated in FIG. 3. In the example illustrated in FIG. 9, the high-frequency waveform signal P1 having the cycle T, the pulse width (period) t1, and the pulse width (period) t2, which are identical to those of the asymmetric waveform electric field illustrated in FIG. 3, is fed to the first electrode 111, and the high-frequency waveform signal P2 having the cycle T, the pulse width (period) t1, and the pulse width (period) t2, which are identical to those of the asymmetric waveform electric field illustrated in FIG. 3, is fed to the second electrode 112. The amplitude of the high-frequency waveform signal P1 is Vmin/2 to −Vmax/2, and the amplitude of the high-frequency waveform signal P2 is Vmax/2 to −Vmin/2. The use of this signal having the asymmetric voltage waveform may also produce the example of the electric field waveform illustrated in FIG. 3.

### Second Embodiment

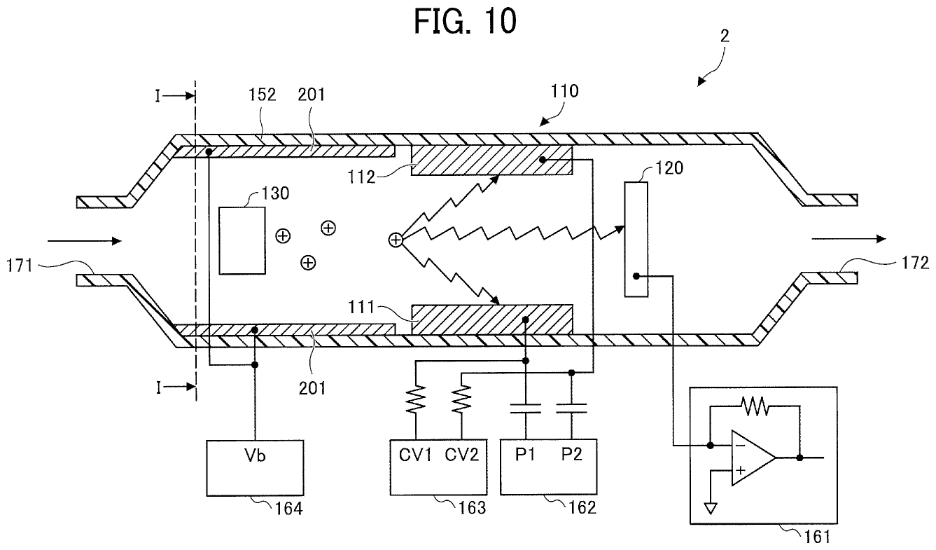

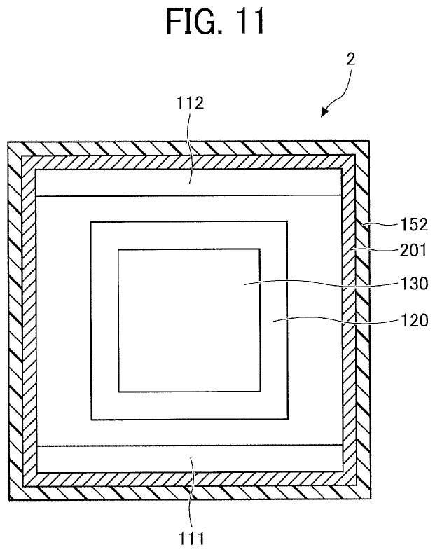

Next, a second embodiment of the present invention is described. The second embodiment relates to an analysis system 2 to which the FAIMS system is applied. FIG. 10 is a schematic view illustrating an example of the configuration of the analysis system 2 according to the second embodiment. FIG. 11 is a cross-sectional view illustrating an example of the configuration of the analysis system 2 according to the second embodiment. FIG. 11 corresponds to the cross-sectional view taken through the line I-I in FIG. 10.

The analysis system 2 according to the second embodiment includes an insulating housing 152 instead of the conductive housing 151. The housing 152 includes, for example, resin. The first electrode 111 and the second electrode 112 are provided on the inner wall surface of the housing 152, and the insulator 113 and the insulator 114 included in the analysis system 1 according to the first embodiment are not included in the analysis system 2. A bias electrode 201 is provided on the inner wall surface of the housing 152 on the side closer to the inlet 171 than the first electrode 111 and the second electrode 112. For example, the bias electrode 201 is formed at least between the ionizer 130 and the first electrode 111 together with the second electrode 112 so as to cover the entire inner wall surface of the housing 152. The bias voltage generation circuit 164 applies the bias voltage Vb not to the housing 152 but to the bias electrode 201. According to the second embodiment, the housing 152 is an example of a frame, and the combination of the housing 152 and the bias electrode 201 may be regarded as a housing. The other structures are the same as those in the first embodiment.

In the analysis system 2 according to the second embodiment, when the ionizer 130 positively ionizes the sample gas, the bias voltage generation circuit 164 applies the positive bias voltage Vb to the bias electrode 201. When the ionizer 130 negatively ionizes the sample gas, the bias voltage generation circuit 164 applies the negative bias voltage Vb to the bias electrode 201. Therefore, the ionized sample gas receives an electric repulsive force from the bias electrode 201 and is unlikely to come into contact with the bias electrode 201 and the housing 152. Thus, according to the second embodiment, the sufficient number of ions may reach the ion detection electrode 120, which may result in an improvement in the detection sensitivity.

In the analysis system 2, the bias electrode 201 may be formed in a annular shape continuous in the circumferential direction. That is, the bias electrode 201 may entirely surround the flow path of the sample gas in four directions.

The bias electrode 201 may be provided at least on the inner wall surface of the housing 152 and between the ionizer 130 and the ion filter 110.

The bias electrode 201 may include, for example, a metallic foil. The bias electrode 201 may be formed by using a metal deposition method or a plating method.

### Third Embodiment

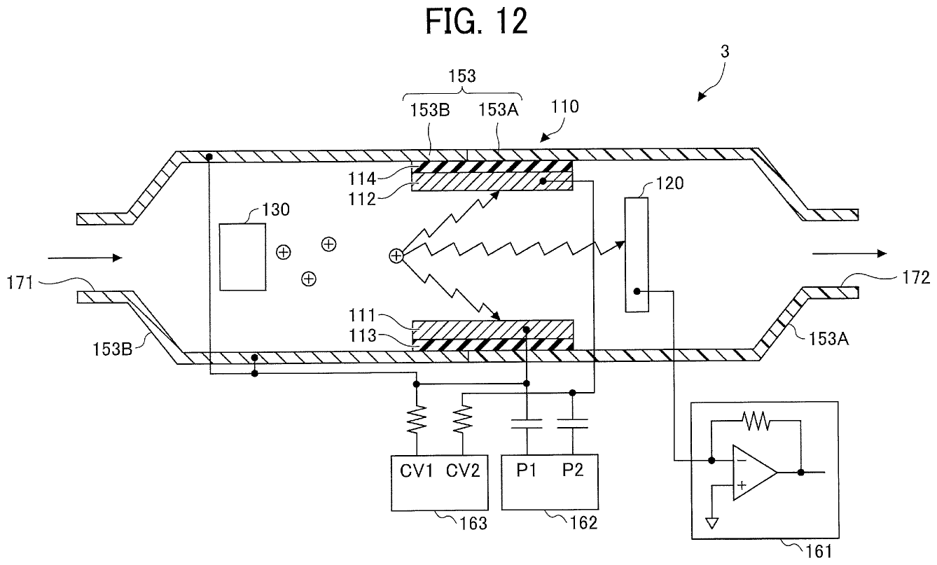

Next, a third embodiment of the present invention is described. The third embodiment relates to an analysis system 3 to which the FAIMS system is applied. FIG. 12 is a schematic view illustrating an example of the configuration of the analysis system 3 according to the third embodiment.

The analysis system 3 according to the third embodiment includes, instead of the housing 151 that is conductive in its entirety, a housing 153 including an insulator 153A having insulating properties on the side of the outlet 172 and a conductor 153B having conductive properties on the side of the inlet 171. The insulator 153A includes, for example, resin. The conductor 153B includes, for example, metal. The compensation voltage generation circuit 163 is coupled to the conductor 153B so as to feed the signal CV1 to the conductor 153B. The analysis system 3 does not include the bias voltage generation circuit 164 included in the analysis system 1. The other structures are the same as those in the first embodiment.

In the analysis system 3 according to the third embodiment, when the ionizer 130 positively ionizes the sample gas, the compensation voltage generation circuit 163 feeds the signal CV1 for a constant positive voltage to the conductor 153B. When the ionizer 130 negatively ionizes the sample gas, the compensation voltage generation circuit 163 applies the signal CV1 for a constant negative voltage to the conductor 153B. Therefore, the ionized sample gas receives an electric repulsive force from the conductor 153B and is unlikely to come into contact with the conductor 153B. Thus, according to the third embodiment, the sufficient number of ions may reach the ion detection electrode 120, which may result in an improvement in the detection sensitivity.

As the signal CV2 includes a compensation voltage and changes in accordance with a code, the signal CV1 may be used as the signal fed to the conductor 153B.

The housing 151 may be used instead of the housing 153, and the compensation voltage generation circuit 163 may be coupled to the housing 151 so as to feed the signal CV1.

In the first embodiment and the second embodiment, the compensation voltage generation circuit 163 may apply the voltage to the housing 151 or the bias electrode 201 as in the third embodiment.

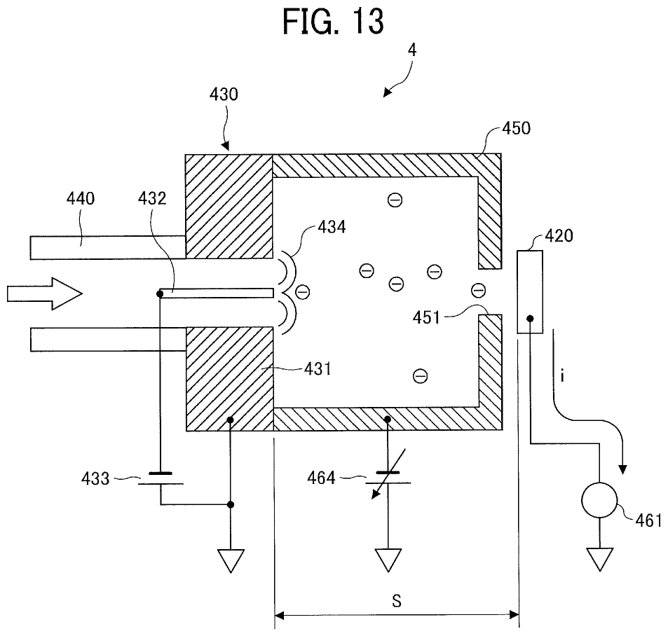

Next, the experiment performed by the inventors of the present application is described. FIG. 13 is a schematic view illustrating a device 4 used in the experiment.

The device 4 used in the experiment includes a housing 450, an introducer 440 that introduces the sample gas into the housing 450, an ionizer 430 disposed between the introducer 440 and the housing 450, and an ion detection electrode 420 located downstream of an outlet 451 of the housing 450. The ionizer 430 includes a cylindrical positive electrode 431 and a needle-shaped negative electrode 432. A DC high-voltage source 433 is coupled between the positive electrode 431 and the negative electrode 432. The ionizer 430 generates negative ions due to corona discharge 434. The housing 450 includes metal. A variable bias voltage source 464 is coupled to the housing 450 so as to apply a negative bias voltage. An ammeter 461 is coupled to the ion detection electrode 420.

After dry air is delivered into the device 4 through the introducer 440, the delivered dry air passes through the ionizer 430 and the housing 450 and is then discharged through the gap between the housing 450 and the ion detection electrode 420. The high-voltage source 433 applies the negative voltage (HV) to the discharge needle (the negative electrode 432) with reference to the ground (GND) electrode to cause the corona discharge so as to generate a negative ion. Ions move along the flow of the dry air in the space inside the housing 450 by a distance S between the ionizer 430 and the ion detection electrode 420 to come into contact with the ion detection electrode 420 and is discharged to the outside. After the ion comes into contact with the ion detection electrode 420, the ion releases the electric charge into the ion detection electrode 420. The released electric charge flows as a current source i and is measured by the ammeter 461. The ammeter 461 uses a current-voltage conversion circuit (IV conversion). The potential of the ion detection electrode 420 is the same as the potential of the GND.

In this experiment, the number of ions generated by the ionizer 430 and reaching the ion detection electrode 420 was measured as an ion current, and the relationship between the bias voltage applied to the housing 450 and the ion current i was examined. Specifically, the dry air was delivered into the device 4 through the introducer 440 at a flow rate of one liter per minute. The high-voltage source 433 applies a voltage of −2.5 kV to the discharge needle (the negative electrode 432). The distance S has two conditions, 10 mm or 20 mm. The bias voltage was changed from 0V to −60V in steps by 10V.

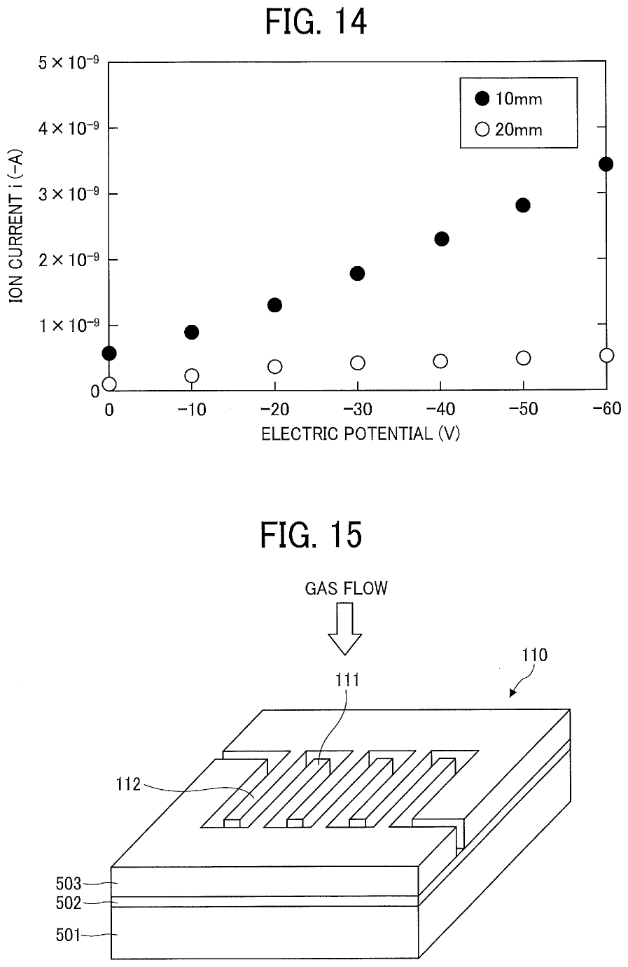

FIG. 14 illustrates the results of the experiment. The horizontal axis in FIG. 14 indicates the bias voltage applied to the housing 450, and the vertical axis indicates the ion current i. The results illustrated in FIG. 14 explain the following two facts.

(1) The shorter the distance S, the larger the ion current i.

(2) The larger the electric potential difference between the ion detection electrode 420 and the housing 450, the larger the ion current i.

The present disclosure uses the feature (2). In the case of negative ions, the housing is set to a negative potential so as to generate an electric field such that ions move toward not the housing but the ion detection electrode at 0 V, which is the GND potential. An increase in the electric potential of the housing at the negative side causes an increase in the electric field acting on ions and thus an increase in the number of ions reaching the ion detection electrode.

Although FIG. 14 illustrates the experimental results with regard to negative ions, the obtained results exhibit the same tendency for positive ions. In this case, the polarity is reversed. A positive voltage is applied to the ionizer to generate positive ions. An increase in the electric potential of the housing at the positive side causes an increase in the electric field acting on ions and thus an increase in the number of ions reaching the ion detection electrode.

The configuration of the first electrode 111 and the second electrode 112 is not limited to parallel flat plates. FIG. 15 is a schematic view illustrating another example of the ion filter 110. For example, as illustrated in FIG. 15, it is possible to use the first electrode 111 and the second electrode 112 that are comb-shaped and are alternately arranged by using a microelectromechanical system (MEMS) process. In this example, a support substrate that is a silicon on insulator (SOI) substrate is used as a support layer 501, an oxide layer is used as an insulating layer 502, and an active layer is used as an electrode layer 503. The first electrode 111 and the second electrode 112 are formed on the electrode layer 503. The support layer 501 may be used as the ion detection electrode 120. In this example, a size reduction may be achieved while an ion passage area is ensured.

The analysis system according to each of the embodiments may be used to, for example, analyze the components of feces odor gas generated by feces. In recent years, the relationship between the state of the bacterial flora in the intestine and the health condition has attracted attention. It is said that several hundreds of types of intestinal bacteria settle down in the human intestine and the intestinal bacteria are roughly classified into good bacteria, bad bacteria, and opportunistic bacteria. Furthermore, it is said that the ideal composition ratio (balance) is “2:1:7”. The balance of these intestinal bacteria is said to change by individual or by age and may serve as a barometer for the health condition. It is said that the disturbance in the dietary habit or the lifestyle habit, stress, constipation, or the like, promotes the growth of bad bacteria, produces gas with a foul odor, and sometimes produces carcinogens. Therefore, in some researches, the components of feces odor gas generated by feces are analyzed to examine the state of the bacterial flora so as to determine the health condition and find a disease at an early stage. The analysis system according to each of the embodiments may be used to analyze the components of feces odor gas as described above. That is, it is possible to configure a feces odor gas analysis system including the analysis system according to any of the embodiments.

The analysis system according to each of the embodiments may be also used to, for example, analyze the components contained in the human exhaled air. In recent years, the relationship between a small amount of exhaled gas components contained in the human exhaled air and a disease has become increasingly clear. An exhaled gas component whose concentration in the exhaled air is correlated with a disease is called a marker substance. The analysis system according to each of the embodiments may be also used to analyze the exhaled gas component. That is, it is possible to configure an exhaled gas analysis system including the analysis system according to any of the embodiments.

The analysis system according to each of the embodiments may be also used for, for example, the analysis to detect the presence or absence of gas harmful to the human body or the analysis of gas generated by matured food.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention.

Any one of the above-described operations may be performed in various other ways, for example, in an order different from the one described above.

Each of the functions of the described embodiments may be implemented by one or more processing circuits or circuitry. Processing circuitry includes a programmed processor, as a processor includes circuitry. A processing circuitry also includes devices such as an application specific integrated circuitry (ASIC), digital signal processor (DSP), field programmable gate array (FPGA), and conventional circuitry components arranged to perform the recited functions.

1. An analysis system comprising:
an ionizer configured to ionize sample gas so as to have a first polarity;
a detection circuit configured to detect at least a part of ions of the sample gas;
a housing accommodating the ionizer and the detection circuit; and
a voltage generation circuit configured to apply a voltage having the first polarity to the housing to generate an electric field inside the housing between the ionizer and the detection circuit, the electric field having a first component in a first direction perpendicular to a second direction parallel to a flow direction of the sample gas and a second component in a third direction perpendicular to the first direction and the second direction.

2. The analysis system according to claim 1, wherein the housing is formed in an annular shape continuous in a circumferential direction of the housing.

3. The analysis system according to claim 1, wherein the housing is conductive.

4. The analysis system according to claim 1, wherein
the housing includes:
a frame having an insulating property; and
an electrode provided inside the frame, and
the voltage generation circuit applies the voltage having the first polarity to the electrode.

5. The analysis system according to claim 1, further comprising an ion filter provided between the ionizer and the detection circuit.

6. The analysis system according to claim 5, wherein the housing has a conductive property at least between the ionizer and the ion filter.

7. The analysis system according to claim 5, wherein the voltage generation circuit applies an offset voltage to an electrode included in the ion filter.

8. A feces odor gas analysis system comprising the analysis system according to claim 1.

9. An exhaled gas analysis system comprising the analysis system according to claim 1.

10. An analysis system comprising:
ionizing means for ionizing sample gas so as to have a first polarity;
detecting means for detecting at least a part of ions of the sample gas;
housing means accommodating the ionizing means and the detecting means; and
voltage applying means for applying a voltage having the first polarity to the housing means to generate an electric field inside the housing means between the ionizing means and the detecting means, the electric field having a first component in a first direction perpendicular to a second direction parallel to a flow direction of the sample gas and a second component in a third direction perpendicular to the first direction and the second direction.
